(19) United States
(12) Patent Application Publication
(10) Pub. No.: US 2023/0172506 A1
(43) Pub. Date: Jun. 8, 2023

(54) FATIGUE ESTIMATION SYSTEM, FATIGUE ESTIMATION METHOD, AND RECORDING MEDIUM

(71) Applicant: Panasonic Intellectual Property Management Co., Ltd., Osaka (JP)

(72) Inventor: Kazuaki HASHIMOTO, Osaka (JP)

(21) Appl. No.: 17/997,462

(22) PCT Filed: May 19, 2021
(86) PCT No.: PCT/JP2021/018944

§ 371 Date: Oct. 28, 2022

(30) Foreign Application Priority Data
May 27, 2020 (JP) 2020-092136

(51) Int. Cl. A61B 5/16; A61B 5/11

(52) U.S. Cl. CPC A61B 5/16; A61B 5/1116

(57) ABSTRACT

A fatigue estimation system estimates the posture of a subject in a predetermined time period based on information output in the predetermined time period, determines whether the estimated posture of the subject matches a specific posture, and estimates, as the fatigue level of the subject accumulated in the predetermined time period, a calculated value obtained by a unit fatigue level being accumulated in accordance with a time period in which the estimated posture of the subject is determined to match the specific posture.

### CROSS-REFERENCE OF RELATED APPLICATIONS

This application is the U.S. National Phase under 35 U.S.C. § 371 of International Patent Application No. PCT/JP2021/018944, filed on May 19, 2021, which in turn claims the benefit of Japanese Patent Application No. 2020-092136, filed on May 27, 2020, the entire disclosures of which Applications are incorporated by reference herein.

### TECHNICAL FIELD

The present disclosure relates to a fatigue estimation system, a fatigue estimation method, and a recording medium for estimating the fatigue level of a subject.

### BACKGROUND ART

In recent years, cases such that the accumulation of fatigue leads to poor health, injuries, accidents, etc. are found here and there. This has brought our attentions to the technique of estimating the level of fatigue to prevent poor health, injuries, accidents, etc. For example, as a fatigue estimation system for estimating a fatigue level that is the level of fatigue, there has been disclosed a fatigue determination device that determines presence or absence of fatigue and the type of the fatigue based on force measurement and bioelectrical impedance analysis (see PTL 1).

### CITATION LIST

### Patent Literature

[PTL 1] Japanese Unexamined Patent Application Publication No. 2017-023311

### SUMMARY OF INVENTION

### Technical Problem

Unfortunately, computing for fatigue level estimation is not performed appropriately in some cases. In view of this, the present disclosure provides, for instance, a fatigue estimation system that estimates the fatigue level of a subject through more appropriate computing.

### Solution to Problem

A fatigue estimation system according to one aspect of the present disclosure includes: an information output device that outputs information regarding locations of body parts of a subject; a storage device which stores postural fatigue information, where the postural fatigue information is information in which a specific posture of the subject is associated with a unit fatigue level, and the unit fatigue level indicates a level of fatigue accumulated in the subject as a result of the subject keeping the specific posture for a unit time; and an estimation device that estimates a fatigue level of the subject in a predetermined time period, where the fatigue level indicates a level of fatigue accumulated in the subject. The estimation device: estimates a posture of the subject in the predetermined time period based on the information output in the predetermined time period; determines whether the estimated posture of the subject matches the specific posture; and estimates, as the fatigue level of the subject in the predetermined time period, a calculated value obtained by the unit fatigue level being accumulated in accordance with a time period in which the estimated posture of the subject is determined to match the specific posture.

A fatigue estimation method according to one aspect of the present disclosure includes: obtaining information regarding locations of body parts of a subject; reading postural fatigue information from a storage device, where the postural fatigue information is information in which a specific posture of the subject is associated with a unit fatigue level, and the unit fatigue level indicates a level of fatigue accumulated in the subject as a result of the subject keeping the specific posture for a unit time; estimating a fatigue level of the subject in a predetermined time period, where the fatigue level indicates a level of fatigue accumulated in the subject; estimating a posture of the subject in the predetermined time period based on the information output in the predetermined time period; determining whether the estimated posture of the subject matches the specific posture; and estimating, as the fatigue level of the subject in the predetermined time period, a calculated value obtained by the unit fatigue level being accumulated in accordance with a time period in which the estimated posture of the subject is determined to match the specific posture.

One aspect of the present disclosure can be implemented as a non-transitory computer-readable recording medium having recorded thereon a program for causing a computer to execute the fatigue estimation method described above.

### Advantageous Effects of Invention

The fatigue estimation system according to one aspect of the present disclosure, for instance, can estimate the fatigue level of a subject through more appropriate computing.

### BRIEF DESCRIPTION OF DRAWINGS

FIG. 1 is a schematic diagram for explaining the overview of a fatigue estimation system according to an embodiment.

FIG. 2 is a block diagram illustrating the functional configuration of the fatigue estimation system according to the embodiment.

FIG. 3 is a diagram for explaining postural fatigue information according to the embodiment.

FIG. 4A is a first diagram for explaining a specific posture according to the embodiment.

FIG. 4B is a second diagram for explaining a specific posture according to the embodiment.

FIG. 4C is a third diagram for explaining a specific posture according to the embodiment.

FIG. 5 is a diagram illustrating a musculo-skeletal model used for constructing postural fatigue information according to the embodiment.

FIG. 6A is a first diagram for explaining a feature according to the embodiment.

FIG. 6B is a second diagram for explaining the feature according to the embodiment.

FIG. 7 is a third diagram for explaining a feature according to the embodiment.

FIG. 8 is a diagram for explaining a blank period according to the embodiment.

FIG. 9 is a diagram illustrating information to be output from the fatigue estimation system according to the embodiment.

FIG. 10 is a flowchart illustrating an operation of the fatigue estimation system according to the embodiment.

### DESCRIPTION OF EMBODIMENTS

Hereinafter, embodiments of the present disclosure will be described with reference to the drawings. Note that each of the embodiments described below illustrates a generic or specific example. Moreover, numerical values, shapes, materials, elements, arrangement and connection of the elements, steps, an order of steps, etc. described in the following embodiments are mere examples and are not intended to limit the present disclosure. Among elements described in the following embodiments, those not recited in any one of the independent claims are described as optional elements.

Note that the drawings are schematic and are not necessarily accurate illustrations. Elements having substantially same configurations are assigned with like reference signs in the drawings, and overlapping description thereof may be omitted or simplified.

### Embodiment

### System Configuration

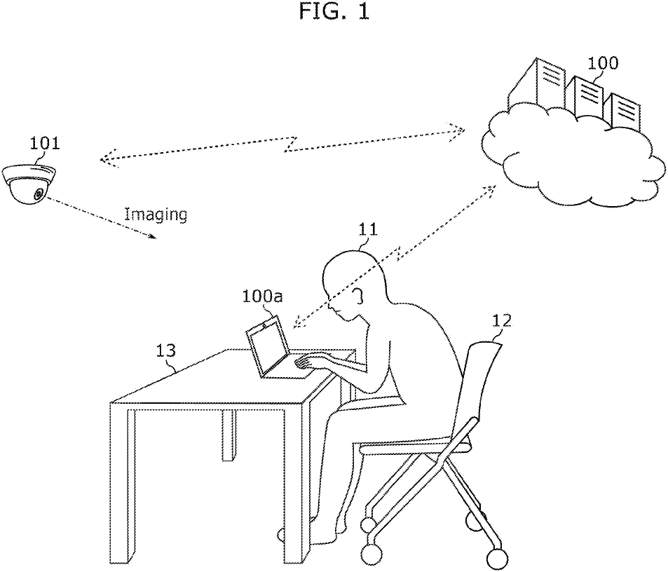

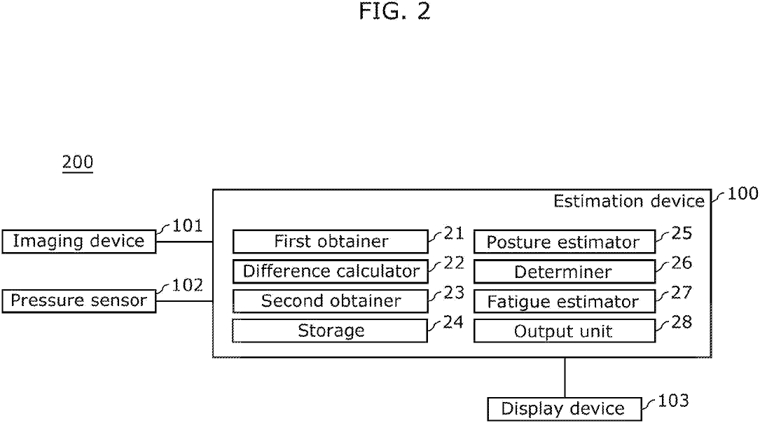

First, the overall configuration of a fatigue estimation system according to an embodiment will be described with reference to FIG. 1 and FIG. 2. FIG. 1 is a schematic diagram for explaining the overview of the fatigue estimation system according to the embodiment. FIG. 1 illustrates how the fatigue level of subject 11 is estimated using fatigue estimation system 200. In the scene illustrated in FIG. 1, subject 11 sits on chair 12 and operates computer 100a placed on desk 13.

In the present embodiment, fatigue estimation system 200 estimates the fatigue level of subject 11 based on images of subject 11 captured by imaging device 101. The images captured by imaging device 101 are transmitted to estimation device 100 via a network such as the Internet. Estimation device 100 is, for example, a computing device mounted on a server device such as a cloud server, and estimates, based on images, the fatigue level of subject 11 included in each of the images. The result of the estimation is, for example, transmitted to computer 100a operated by subject 11 via a network, and displayed on the screen of computer 100a.

In this way, subject 11 can check, while working using computer 100a, an estimation result displayed on the same computer 100a. Note that the present embodiment illustrates an example in which estimation device 100 is implemented by a server device, as described above, but the configuration of fatigue estimation system 200 is not limited to such an example. For example, estimation device 100 may be built into computer 100a. In other words, computer 100a is an estimation device in another embodiment.

In the present disclosure, when estimation device 100 estimates the fatigue level of subject 11 from the posture of subject 11, it is possible to significantly reduce the amount of computing by using postural fatigue information constructed in advance. The details of the postural fatigue information will be described later. In view of the above, fatigue estimation system 200 can be implemented even through computing performed using, for example, computer 100a with low processing performance.

When using computer 100a as an estimation device, it is possible to implement fatigue estimation system 200 with a simple configuration since there is no need for fatigue estimation system 200 to include a network and a server device. Computer 100a may be provided with a camera at a position that enables capturing images of subject 11, and by using the camera as imaging device 101 described above, it is also possible to implement fatigue estimation system 200 with the use of computer 100a alone.

If fatigue estimation system 200 is implemented using a server device with relatively high processing performance, it is possible to obtain an estimation result approximately at the same time as image capturing performed by imaging device 101. Subject 11 can therefore work while being aware of his/her fatigue level at all times.

FIG. 2 is a block diagram illustrating the functional configuration of the fatigue estimation system according to the embodiment. As illustrated in FIG. 2, fatigue estimation system 200 according to the embodiment includes estimation device 100, imaging device 101, pressure sensor 102, and display device 103.

As described above, estimation device 100 is a processing device that estimates a fatigue level indicating the level of fatigue accumulated in subject 11, and is implemented by being mounted on a server device. Estimation device 100 includes first obtainer 21, difference calculator 22, second obtainer 23, storage 24, posture estimator 25, determiner 26, fatigue estimator 27, and output unit 28.

First obtainer 21 is a communication module that obtains images in each of which subject 11 is captured. For example, first obtainer 21 obtains images captured by imaging device 101 by communicating with imaging device 101 via a network.

Imaging device 101 is a device that outputs images each including subject 11 by capturing the images, and is implemented by a camera installed in a facility, such as a security camera, or a camera built into, for instance, computer 100a or a mobile device, or a dedicated camera for use in fatigue estimation system 200. Note that images output by imaging device 101 and obtained by first obtainer 21 are so-called a video sequentially captured in time series. First obtainer 21 obtains such a video in parallel to image capturing performed by imaging device 101. First obtainer 21 outputs the obtained images to posture estimator 25.

Posture estimator 25 is a processing unit that estimates the posture of subject 11 based on images output from first obtainer 21. Posture estimator 25 is implemented by a predetermined program being executed by, for instance, a processor and memory. As described above, since the images are a video composed by a sequence of images in time series, posture estimator 25 estimates the posture of subject 11 for each of the images composing the video. Accordingly, the estimated postures of subject 11 are output from posture estimator 25 through the entire time period in which fatigue level estimation is performed. Note, however, that when subject 11 is outside the field of view of imaging device 101, posture estimator 25 may stop estimating the posture of subject 11.

Posture estimator 25 localizes the joint positions of subject 11 in an image by image processing performed using a predetermined program. Posture estimator 25 outputs, as the result of the posture estimation, a joint position model expressed by connecting two joints by a bone having a predetermined length based on the relative positions of the joints. Note that the joint position model may be read as a skeletal position model since the relative positions of joints are in one-to-one correspondence with the relative positions of bones connecting the joints. Estimation device 100 estimates the fatigue level of subject 11 by performing matching between the joint position model output as the posture of subject 11 and the postural fatigue information stored in storage 24.

Storage 24 is a storage device implemented by, for instance, a semiconductor memory, a magnetic storage medium, or an optical storage medium. Storage 24 stores various types of information including postural fatigue information for use in estimation device 100. Each of processing units in estimation device 100 reads necessary information from storage 24 to use the information, and if necessary, newly writes information generated or the like into storage 24.

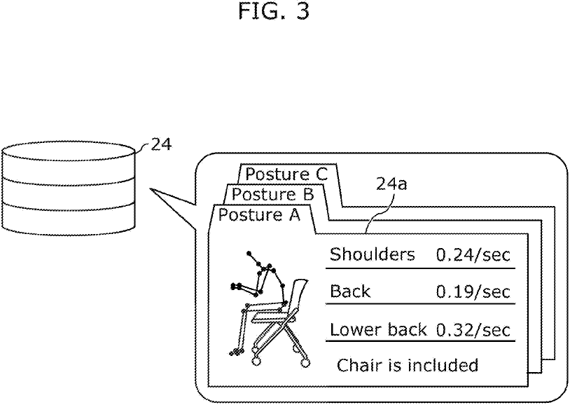

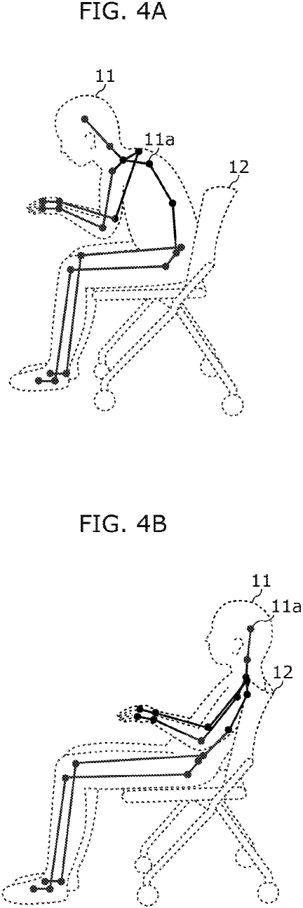

The postural fatigue information will be described with reference to FIG. 3, and FIG. 4A through FIG. 4C. FIG. 3 is a diagram for explaining postural fatigue information according to the embodiment. FIG. 3 illustrates storage 24 and postural fatigue information stored in storage 24. The postural fatigue information is information that associates a specific posture with unit fatigue levels each indicating the level of fatigue accumulated in subject 11 as a result of subject 11 keeping the specific posture for a unit time.

In the present embodiment, the postural fatigue information includes information on a plurality of specific postures (three here), and the plurality of specific postures are referred to as posture A, posture B, and posture C for the sake of convenience. FIG. 4A is a first diagram for explaining a specific posture according to the embodiment, and subject 11 taking posture A described above is indicated by dashed lines. FIG. 4B is a second diagram for explaining a specific posture according to the embodiment, and subject 11 taking posture B described above is indicated by dashed lines. FIG. 4C is a third diagram for explaining a specific posture according to the embodiment, and subject 11 taking posture C is indicated by dashed lines. Note that the postural fatigue information may include many more specific postures.

For example, information 24a on posture A included in the postural fatigue information is a specific posture corresponding to the posture of subject 11 illustrated in FIG. 4A. As illustrated in FIG. 3 and FIG. 4 through FIG. 4C, a specific posture in the postural fatigue information is defined by the relative positions of joints (or bones), where the joints of subject 11 indicated by black dots are connected by the bones of subject 11 indicated by straight lines. In other words, a specific posture in the postural fatigue information is joint position model 11a having information equivalent to the output of posture estimator 25 described above. As illustrated in FIG. 3, the postural fatigue information indicates unit fatigue levels of subject 11 accumulated per unit time (one second here).

In the present embodiment, different fatigue levels are set for the unit fatigue levels of posture A depending on a part of subject 11. Specifically, the following unit fatigue levels indicating the levels of fatigue accumulated as a result of subject 11 keeping posture A for a unit time are individually set for posture A: 0.24 is set for a first unit fatigue level indicating the level of fatigue accumulated in the shoulders that are a first part of subject 11, 0.19 is set for a second unit fatigue level indicating the level of fatigue accumulated in the back that is a second part of subject 11, and 0.32 is set for a third unit fatigue level indicating the level of fatigue accumulated in the lower back that is a third part of subject 11. In other words, if subject 11 keeps posture A, different fatigue levels are accumulated per second for the shoulders, back, and lower back of subject 11.

Since posture A according to the present embodiment is a posture presupposing a sitting posture on chair 12, whether a chair is present in the vicinity of subject 11 is determined when matching is performed between posture A and the posture of subject 11. Information 24a on posture A includes, for use in the detection of a chair, information indicating that chair 12 is present in the vicinity of subject 11. Note that the vicinity of subject 11 is a range that allows subject 11 to be in contact with an object and that physically contributes to subject 11′s keeping of a posture by the presence of an object such as chair 12. The vicinity of subject 11 indicates, for example, a range including a location with which the hands and feet of subject 11 come in contact when stretched.

In this way, a specific posture may be defined as a posture kept by intervention of an object such as chair 12 or desk 13. There are opposite cases where a specific posture is defined as a posture kept without intervention of an object. When subject 11 is in a standing posture, for example, it is difficult, with the use of a joint position model, to predict whether subject 11 is holding a package or the like. Since the fatigue level of subject 11 largely varies depending on the presence or absence of a package held, it is necessary to differentiate the case where subject 11 is holding a package or the like from the case where subject 11 is not holding any package.

Accordingly, with the above configuration, posture estimator 25 can estimate that subject 11 merely keeps a standing posture when a package or the like is not present as an object in the vicinity of subject 11. In the present embodiment, since the specific posture of subject 11 is thus defined by the presence or absence of an object in the vicinity of subject 11 and object-dependent fatigue level differentiation is thereby allowed, it is possible to perform more accurate fatigue level estimation.

Note that the detection of an object in the vicinity of subject 11 is performed based on images obtained from imaging device 101. In other words, it can be said that images obtained by first obtainer 101 include both information to be used by posture estimator 25 for estimating the posture of subject 11 and object detection information indicating the presence or absence of an object in the vicinity of subject 11. Computing of the fatigue level of subject 11 with the use of the object detection information will be described later together with the description of determiner 26 and fatigue estimator 27.

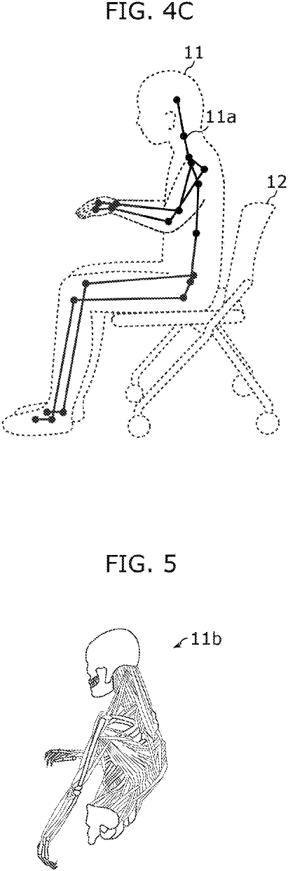

Next, a method for constructing postural fatigue information as described above will be described with reference to FIG. 5. FIG. 5 is a diagram illustrating a musculo-skeletal model used for the construction of the postural fatigue information according to the embodiment. The posture (estimated posture) of subject 11 that is output as a joint position model is reproduced as joint position model 11b through analytical processing such as forward dynamics analysis or backward dynamics analysis using musculo-skeletal model 11b as illustrated in FIG. 5. By reproducing a certain posture with the use of musculo-skeletal model 11b, it is possible to quantify a load imposed on joints and muscles due to subject 11 keeping that posture. When the posture of musculo-skeletal model 11b is kept for a certain time, it is possible to quantify degradation in blood flow on the model.

Accordingly, by reproducing the estimated posture using musculo-skeletal model 11b, it is possible to obtain, through calculation, values indicating muscle loading, joint loading, and the degree of degradation in blood flow after a certain time. Since muscle loading, joint loading, and the degree of degradation in blood flow are closely related to fatigue, it is possible, with the use of these values, to quantify a fatigue level accumulated per certain time (i.e., the unit time described above) in keeping the estimated posture. Calculation for the quantification is, however, not realistic because it requires a huge amount of computing and therefore requires time and processing performance to apply such computing to all of estimated postures.

In the present embodiment, it is possible to omit the computing described above by performing computing for each of specific postures in advance and using postural fatigue information in which a specific posture is directly associated with fatigue levels. Accordingly, it is possible to immediately estimate the fatigue level of subject 11 based on whether the estimated posture matches any one of the specific postures. In the present embodiment, it is therefore possible to achieve fatigue level estimation using estimation device 100 with low processing performance and enhance immediacy in the fatigue level estimation, thereby estimating the fatigue level of a subject through more appropriate computing.

The specific posture here is defined as a posture having an allowed range including: a reference posture serving as a reference; and a posture whose locations of joints are deviated from those of the reference posture within a predetermined range. This allows an inclusion of a plurality of estimated postures under a single specific posture, thereby enabling easier calculation. Widening the allowed range of a specific posture, however, might degrade the accuracy of a fatigue level to be estimated. Narrowing the allowed range of a specific posture, on the other hand, might create a “hole” representing postures that cannot be estimated. And yet, constructing postural fatigue information to include a large number of specific postures for filling such a “hole” significantly increases the amount of information and increases a processing cost required for matching an estimated posture and each of the specific postures.

Accordingly, the allowed range of a specific posture may be set in accordance with the accuracy of a fatigue level to be estimated which is required by a manager or the like who sets up fatigue estimation system 200. Note that estimation device 100 may, for example, merely select a specific posture that is the closest to an estimated posture from among specific postures included in postural fatigue information, and accumulate unit fatigue levels associated with the selected specific posture. Alternatively, when the estimated posture does not match any of the specific postures included in the postural fatigue information, for example, estimation device 100 may allocate an average fatigue level as a unit fatigue level and estimate the fatigue level of the posture that does not match any of the specific postures.

In the present embodiment, more accurate fatigue level estimation is performed by correcting unit fatigue levels using a feature of an estimated posture. For example, the difference (i.e., the amount of deviation) between an estimated posture and a reference posture serving as a reference for a specific posture that the estimated posture matches is used as a feature of the estimated posture, to correct unit fatigue levels that are set for the specific posture. The correction is performed mainly by difference calculator 22 and second obtainer 23.

Referring back to FIG. 2, difference calculator 22 is a processing unit that calculates the difference between a reference posture and an estimated posture. Difference calculator 22 is implemented by a predetermined program being executed by, for instance, a processor and memory. Specifically, difference calculator 22 uses a specific posture found as a result of matching between a specific posture in postural fatigue information and a posture estimated by determiner 26 and fatigue estimator 27 which are to be described later. The specific posture includes a reference posture, as described above, and difference calculator 22 calculates the difference between the estimated posture and the reference posture.

Second obtainer 23 is a processing unit that obtains an amount of deviation which is a calculated feature, and corrects unit fatigue levels. Second obtainer 23 is implemented by a predetermined program being executed by, for instance, a processor and memory.

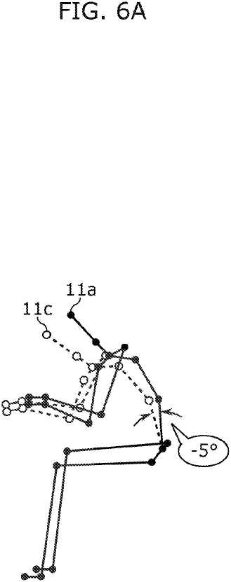

Hereinafter, the details of operations performed by difference calculator 22 and second obtainer 23 will be described with reference to FIG. 6A and FIG. 6B. FIG. 6A is a first diagram for explaining a feature according to the embodiment. In FIG. 6A, an estimated posture is indicated by joint position model 11c presented by dashed lines and white circles, whereas a reference posture is indicated by joint position model 11a presented by solid lines and black circles. FIG. 6B is a second diagram for explaining the feature according to the embodiment. FIG. 6B shows the correlation between (i) the difference (here, an angular difference in the upper half of the body with waist joints serving as an axis) between the reference posture and the estimated posture, and (ii) a unit fatigue level to be determined through correction.

For example, difference calculator 22 may calculate a difference regarding a body part having a dominant unit fatigue level when the unit fatigue level of a specific posture which an estimated posture matches is separated into unit fatigue levels each corresponding to a different one of body parts. In other words, in posture A described above, the unit fatigue level of the lower back indicates the largest value and is thus a dominant unit fatigue level. In view of this, in the present embodiment, the difference between the reference posture and the estimated posture is calculated, focusing on the waist joints, as illustrated in FIG. 6A. The case here is a case where the waist joints serve as an axis of rotation and difference calculator 22 calculates, as the difference, an amount of rotation from the reference posture to the estimated posture on the upper body side above the waist joints.

In the example illustrated in FIG. 6A, for example, the estimated posture has, as the difference, an angular difference of -5 degrees relative to the reference posture. Note that a positive or negative sign used herein is assigned, for the sake of convenience, to a value as a sign indicating the direction of deviation, and a negative sign may be replaced with a positive sign or vice versa. In FIG. 6A, a negative sign indicates a forward deviation relative to the reference posture and a positive sign indicates a backward deviation relative to the reference posture.

Second obtainer 23 calculates, using the angular difference, corrected unit fatigue levels based on the correlation illustrated in FIG. 6B. In FIG. 6B, for example, in a difference with a negative sign, the unit fatigue level of the estimated posture increases compared with that of the reference posture (the arrow pointing upward in the figure). In a difference with a positive sign, the unit fatigue level of the estimated posture decreases compared with that of the reference posture (the arrow pointing downward in the figure). Even when absolute values indicate the same difference, an amount of correction may vary depending on whether the sign of a value is positive or negative. In other words, the relationship between the difference and the unit fatigue level is not necessarily linear.

In the present embodiment, a pressure value obtained from pressure sensor 102 is also used as a feature of the posture of subject 11.

Pressure sensor 102 is a detector that has a pressure-sensitive surface and detects pressure imposed on the pressure-sensitive surface as well as the magnitude of the imposed pressure (i.e., a pressure value). The pressure-sensitive surface of pressure sensor 102 is placed, for example, on the seat and backrest of chair 12 which subject 11 sits on, the floor which the feet of subject 11 are in contact with, as well as the top of desk 13 which subject 11 places his/her hands on.

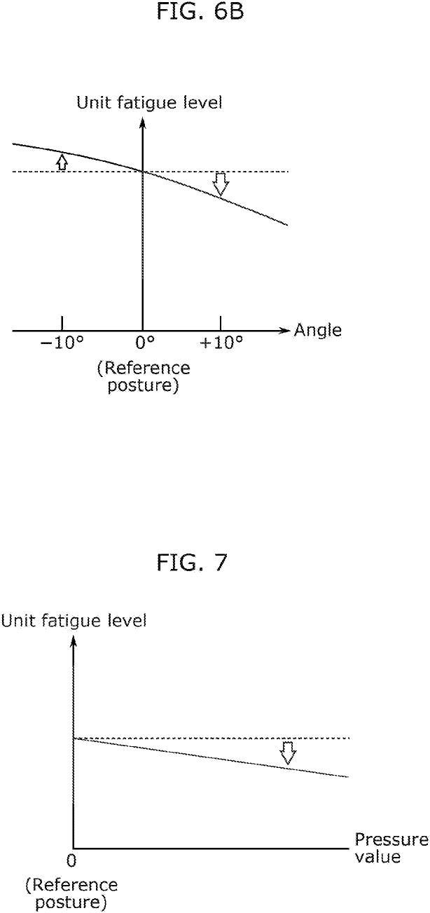

In the present embodiment, second obtainer 23 obtains, from pressure sensor 102, a pressure value indicating pressure imposed on the pressure-sensitive surface by communicating with pressure sensor 102. Second obtainer 23 corrects a unit fatigue level using, as a feature of the posture of subject 11, the pressure value obtained from pressure sensor 102. FIG. 7 is a third diagram for explaining a feature according to the embodiment. FIG. 7 illustrates one example of the correlation between the obtained pressure value and the corrected unit fatigue level.

The correlation between a pressure value and a unit fatigue level may be a positive correlation or a negative correlation in accordance with a body part corresponding to the location of contact with the pressure-sensitive surface of pressure sensor 102. For example, if a pressure value indicating pressure imposed on the top of desk 13 is large, it is assumed that the hands of subject 11 are placed on desk 13, and the correlation indicates a negative correlation in which the fatigue level of the shoulders of subject 11 decreases as the pressure value increases. For example, if a pressure value indicating pressure imposed on the front part of the seat of chair 12 is large, it is assumed that the posture of subject 11 is leaning forward, and the correlation is a positive correlation in which the fatigue level of the lower back of subject 11 increases as the pressure value increases.

Besides the cases described above, second obtainer 23 may: correct the unit fatigue levels of the back and the lower back of subject 11 using pressure values detected by pressure sensor 102 whose pressure-sensitive surface is placed on the backrest of chair 12; and correct the unit fatigue level of the lower part of the body such as legs using a pressure value detected by pressure sensor 102 whose pressure-sensitive surface is placed on the floor. Alternatively, second obtainer 23 may obtain a plurality of pressure values detected by a plurality of pressure sensors 102 whose pressure-sensitive surfaces are placed at different locations, and use the obtained pressure values in combination.

Referring back to FIG. 2, determiner 26 is a processing unit that determines whether an estimated posture matches a specific posture. Determiner 26 is implemented by a predetermined program being executed by, for instance, a processor and memory. Determiner 26 thus performs matching between a specific posture included in postural fatigue information and the estimated posture. When determining that the estimated posture matches the specific posture, determiner 26 outputs an addition command to fatigue estimator 27 so that fatigue estimator 27 accumulates unit fatigue levels associated with the specific posture.

Fatigue estimator 27 is a processing unit that generates a result of accumulating unit fatigue levels, as the fatigue level of subject 11. Fatigue estimator 27 is implemented by a predetermined program being executed by, for instance, a processor and memory. For example, fatigue estimator 27 accumulates unit fatigue levels only in a time period in which fatigue estimator 27 obtains an addition command. Accordingly, the unit fatigue levels are accumulated only for a time period in which the estimated posture is determined to match the specific posture, and the fatigue level accumulated in a time period in which subject 11 keeps the specific posture can be thus estimated.

After estimating the fatigue level of subject 11 in a predetermined time period, fatigue estimator 27 outputs the result of the estimation to an external device via output unit 28. The predetermined time period used herein may be a time period determined in advance, e.g., a day, or a unit time that is the shortest time period per which the fatigue level of subject 11 is updated in the system configuration. Fatigue estimator 27 may output the latest fatigue level resulting from the fatigue level being updated every time the unit time elapses, and reset a cumulative value at a time point when a day has passed. This enables subject 11 to easily grasp the fatigue level accumulated from the start of the day until present.

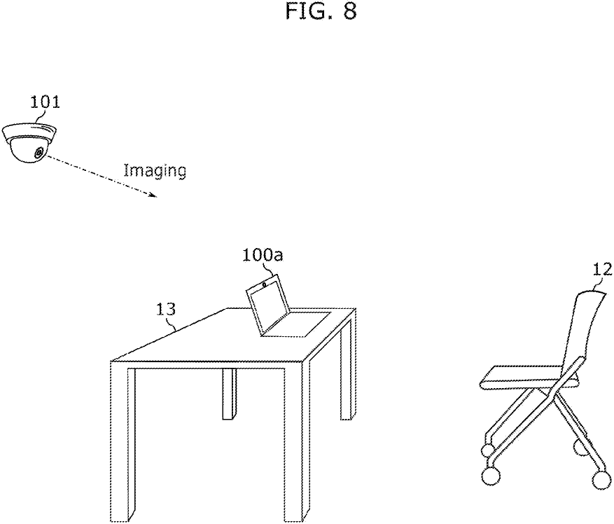

Note that subject 11 is not always present in an area where images can be captured by imaging device 101 during a day, for example. FIG. 8 is a diagram for explaining a blank period according to the embodiment. For example, when subject 11 leaves the desk and gets out of the field of view of imaging device 101, as illustrated in FIG. 8, a blank period in which no image that includes subject 11 can be captured (output) is generated. In such a case, for example, if other imaging device is provided at a location to which subject 11 has moved, fatigue estimation system 200 may obtain images from the other imaging device.

Fatigue estimation system 200 may work together with a scheduling system that manages the schedule of subject 11, to accumulate a preset supplementary fatigue level in accordance with the length of a blank period, based on a predictable reason why subject 11 has left the desk, and add the resultant cumulative value to the fatigue level estimated by fatigue estimator 27. For example, when subject 11 has left the desk in order to take a break or the like, a supplementary fatigue level indicating a negative value may be accumulated in accordance with the length of a blank period, and the resultant cumulative value may be added to the estimated fatigue level. When subject 11 has left the desk to work or the like, for example, a supplementary fatigue level indicating a positive value may be accumulated in accordance with the length of a blank period, and the resultant cumulative value may be added to the estimated fatigue level. Accordingly, it is possible to supplement fatigue levels for a blank period based on the action of subject 11, thereby estimating the fatigue level of subject 11 more accurately even though a blank period is generated.

Output unit 28 is a processing unit that outputs an estimation result including an estimated fatigue level. Output unit 28 obtains the fatigue level of subject 11 estimated by fatigue estimator 27, generates image data including other information, and transmits the image data to display device 103 via a network.

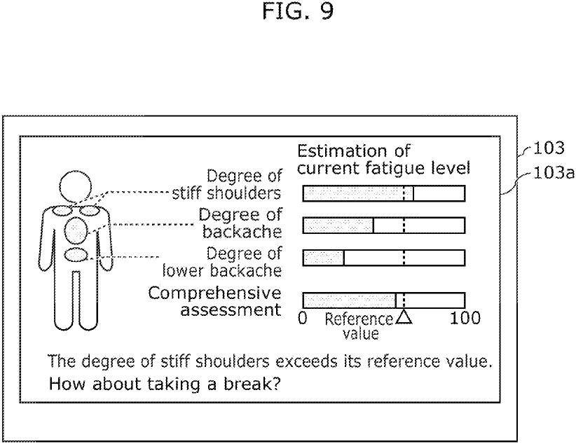

Display device 103 displays image data that has been received. FIG. 9 is a diagram illustrating information to be output from fatigue estimation system 200 according to the embodiment. Display device 103 is a display having display module 103a such as a liquid crystal panel, and displays the received image data by driving display module 103a.

In FIG. 9, image data indicating the current fatigue level of subject 11 is displayed, for example. In the image data, the current fatigue level of subject 11 is indicated separately for each of body parts, as illustrated in FIG. 9. Specifically, “degree of stiff shoulders” indicating the fatigue level of the shoulders of subject 11, “degree of backache” indicating the fatigue level of the back of subject 11, and “degree of lower backache” indicating the fatigue level of the lower back of subject 11 are individually shown in the image data. Additionally, the locations of the body parts in the figure of a person for which the fatigue levels are indicated, comprehensive assessment on the fatigue levels, comment and advice on the result of the fatigue level estimation, etc. are indicated as supplementary information in the image data.

A display included in computer 100a of subject 11 is used for display device 103, as described above, but a different display may be used instead. For example, a dedicated display for fatigue estimation system 200 may be used for display device 103.

### Operation

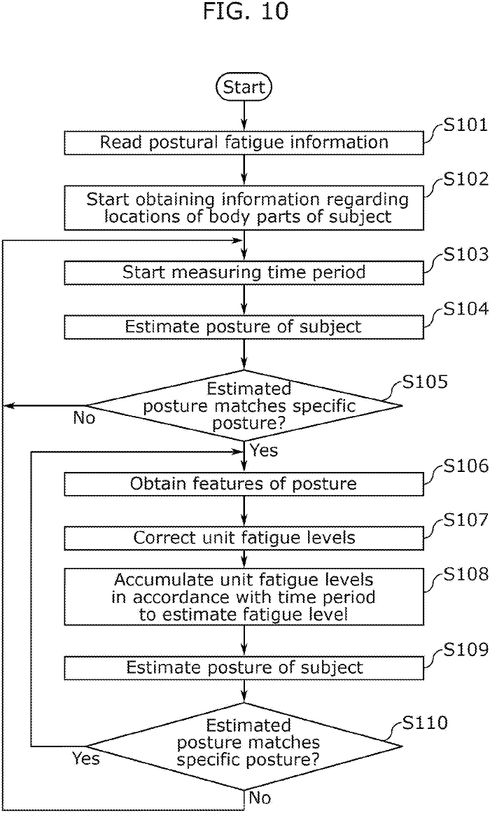

Next, an operation of fatigue estimation system 200 described above will be described with reference to FIG. 10. FIG. 10 is a flowchart illustrating the operation of fatigue estimation system 200 according to the embodiment.

In fatigue estimation system 200 according to the present embodiment, first, posture estimator 25 reads postural fatigue information stored in storage 24 (reading in step S101). The postural fatigue information read is information in which a specific posture is associated with unit fatigue levels.

Imaging device 101 starts operating in advance, and images composing a video are sequentially output from imaging device 101. First obtainer 21 starts obtaining the images output (obtaining in step S102) and continues sequentially obtaining the images until fatigue estimation system 200 is stopped.

Estimation device 100 starts measuring a time period from timing which is a starting point and at which images are started to being obtained (step S103). Posture estimator 25 estimates the posture of subject 11 based on the obtained images (step S104). Determiner 26 determines whether the posture of subject 11 estimated by posture estimator 25 matches a specific posture included in the postural fatigue information (step S105). Note that when a plurality of specific postures are included in the postural fatigue information, the determination is sequentially performed for each of the specific postures.

When the estimated posture does not match the specific posture or the plurality of specific postures do not include a specific posture which the estimated posture matches (No in step S105), estimation device 100 returns to step S103 and starts measuring a time period from different timing. Posture estimator 25 estimates the posture of subject 11 in a time period starting from the different timing (step S104). In this way, the time period measurement and the posture estimation are repeated until the estimated posture of subject 11 matches a specific posture.

When the estimated posture matches the specific posture or the plurality of specific postures include the specific posture which the estimated posture matches (Yes in step S105), difference calculator 22 calculates the difference between a reference posture and the estimated posture of subject 11, and pressure sensor 102 detects and outputs a pressure value. Second obtainer 23 obtains the calculated difference and the detected pressure value as features of the estimated posture of subject 11 (step S106).

Second obtainer 23 corrects the unit fatigue levels of the specific posture which the estimated posture matches, using the obtained features (step S107). Fatigue estimator 27 estimates the fatigue level of subject 11 by accumulating the corrected unit fatigue levels in accordance with the measured time period (i.e., a time period in which the posture matching the specific posture is kept) (step S108). Note that steps S105 through S108 are also referred to as estimating a fatigue level indicating the level of fatigue accumulated in subject 11.

Subsequently, posture estimator 25 estimates the posture of subject 11 (step S109). Determiner 26 determines again whether the estimated posture of subject 11 matches the same specific posture as the one determined to have matched the estimated posture in step S105 (step S110). Accordingly, whether the posture matching the specific posture has been kept is determined.

When the estimated posture of subject 11 matches the specific posture (Yes in S110), estimation device 100 returns to step S106 and performs again the obtainment of the features. The features may vary due to a slight change in the posture even when the estimated posture matches the same specific posture. It is therefore possible to more accurately grasp a change in the fatigue level due to a change in the posture, by performing again the obtainment of the features. Estimation device 100 thus repeats steps S106 through S110 until the posture of subject 11 no longer matches the specific posture, and continues estimating the fatigue level of subject 11 indicating the level of fatigue accumulated in subject 11 with the extension of the time period (i.e., the elapse of time).

When the estimated posture of subject 11 does not match the specific posture, estimation device 100 returns to step S103 and starts measuring a time period from different timing. Posture estimator 25 estimates the posture of subject 11 in a time period starting from the different timing (step S104). The same process is repeated thereafter. Estimation device 100 ends the operation after a predetermined time period set in advance has elapsed.

Note that after the estimated posture is once determined as not matching the specific posture in step S110, determined Yes again in step S105, and then reaches step S108, the fatigue level newly estimated in step S108 after step S105 is added to the fatigue level estimated in step S108 that is before step S110, to estimate the fatigue level in total. Accordingly, it is possible to estimate the fatigue level of subject 11 accumulated over a predetermined time period while subject 11 was changing his/her posture.

It is thus possible, through appropriate computing, to estimate a fatigue level indicating the level of fatigue accumulated in subject 11 without needing to perform complicated computing that uses musculo-skeletal models or the like for each estimated posture.

### Advantageous Effects, Etc.

As described above, fatigue estimation system 200 according to the present embodiment includes: an information output device (imaging device 101, for instance) that outputs information regarding the locations of the body parts of subject 11; storage 24 which stores postural fatigue information, where the postural fatigue information is information in which a specific posture of the subject is associated with a unit fatigue level, and the unit fatigue level indicates the level of fatigue accumulated in the subject as a result of the subject keeping the specific posture for a unit time; and estimation device 100 that estimates the fatigue level of the subject in a predetermined time period, where the fatigue level indicates the level of fatigue accumulated in subject 11. Estimation device 100: estimates the posture of the subject in the predetermined time period based on the information output from the information output device in the predetermined time period; determines whether the estimated posture of subject 11 matches the specific posture; and estimates and outputs, as the fatigue level of subject 11 in the predetermined time period, a calculated value obtained by the unit fatigue level being accumulated in accordance with a time period in which the estimated posture of subject 11 is determined to match the specific posture.

In such fatigue estimation system 200, fatigue levels of subject 11 to be accumulated as a result of subject 11 keeping an estimated posture are stored as postural fatigue information in which the fatigue levels are associated with a specific posture in advance. In general, estimating the fatigue level of a subject from his/her posture requires a huge amount of computing. With fatigue estimation system 200, however, it is possible, by merely referring to the postural fatigue information, to estimate the fatigue level of a subject from his/her posture based on association between a posture and fatigue levels that have been calculated in advance. Since such computing can be achieved also by using, for example, a system with low processing performance, it is possible to implement fatigue estimation system 200 using a simple configuration. When using a system with high processing performance, on the other hand, it is possible to immediately estimate the fatigue level of a subject from his/her posture. It is therefore possible to implement fatigue estimation system 200 that can perform real-time feedback to subject 11 or the like. Accordingly, owing to the review of computing, it is possible to estimate the fatigue level of a subject through appropriate computing in accordance with a system.

For example, in the postural fatigue information: the specific posture of subject 11 may be associated with a first unit fatigue level that is part of the unit fatigue level, where the first unit fatigue level indicates the level of fatigue accumulated in a first part among the body parts of subject 11 as a result of subject 11 keeping the specific posture for a unit time; and the specific posture of subject 11 may be associated with a second unit fatigue level that is part of the unit fatigue level, where the second unit fatigue level indicates the level of fatigue accumulated in a second part among the body parts of subject 11 as a result of subject 11 keeping the specific posture for a unit time. Estimation device 100 may: estimate, as the fatigue level of the first part of subject 11 in the predetermined time period, a calculated value obtained by the first unit fatigue level being accumulated in accordance with the time period in which the estimated posture of subject 11 is determined to match the specific posture; and estimate, as the fatigue level of the second part of subject 11 in the predetermined time period, a calculated value obtained by the second unit fatigue level being accumulated in accordance with the time period in which the estimated posture of subject 11 is determined to match the specific posture.

According to the above features, it is possible to estimate a fatigue level separately accumulated for each of the body parts of subject 11. Even in such estimation, it is possible to individually estimate the fatigue level of subject 11 for each of the body parts through simple computing without requiring a huge amount of computing which is generally required. Accordingly, it is possible to estimate the fatigue level of a subject through appropriate computing.

For example, when determining that the estimated posture of the subject matches the specific posture, estimation device 100 may: obtain a feature of the estimated posture of subject 11; correct the unit fatigue level using the feature; and estimate, as the fatigue level of subject 11 in the predetermined time period, a calculated value obtained by the corrected unit fatigue level being accumulated in accordance with the time period in which the estimated posture of subject 11 is determined to match the specific posture.

According to the above features, it is possible, with the use of a feature of the estimated posture of subject 11, to enhance the accuracy of the fatigue level of subject 11 to be estimated. Even in such estimation, it is possible to more accurately estimate the fatigue level of subject 11 through simple computing without requiring a huge amount of computing which is generally required. Accordingly, it is possible to estimate the fatigue level of a subject through appropriate computing.

For example, the feature may be a pressure value obtained from pressure sensor 102 with which a body part of subject 11 corresponding to the first part of subject 11 is in contact, and estimation device 100 may correct the first unit fatigue level with a greater amount of correction as the pressure value increases.

According to the above features, it is possible, by using a pressure value as a feature of the estimated posture of subject 11, to enhance the accuracy of the fatigue level of subject 11 to be estimated. Even in such estimation, it is possible to more accurately estimate the fatigue level of subject 11 through simple computing without requiring a huge amount of computing which is generally required. Accordingly, it is possible to estimate the fatigue level of a subject through appropriate computing.

For example, the feature may be the difference between the estimated posture of subject 11 and a reference posture included in a range within which the specific posture falls, and the unit fatigue level may be corrected with a greater amount of correction as the difference increases.

According to the above features, it is possible, by using the difference between a reference posture and the estimated posture of subject 11 as a feature of the estimated posture, to enhance the accuracy of the fatigue level of subject 11 to be estimated. Even in such estimation, it is possible to more accurately estimate the fatigue level of subject 11 through simple computing without requiring a huge amount of computing which is generally required. Accordingly, it is possible to estimate the fatigue level of a subject through appropriate computing.

For example, in the postural fatigue information, the specific posture of subject 11 may be defined as a posture kept by intervention of an object. Estimation device 100 may: obtain object detection information indicating presence or absence of the object; and when the object detection information indicates that the object is present, determine whether the estimated posture of subject 11 matches the specific posture.

According to the above features, it is possible to distinguish between a posture that is kept by intervention of an object and a posture that is kept without intervention of an object, to determine whether the estimated posture matches a specific posture. Accordingly, it is possible to estimate the fatigue level of a subject through appropriate computing.

For example, in the postural fatigue information, the specific posture of subject 11 may be defined as a posture kept without intervention of an object. Estimation device 100 may: obtain object detection information indicating presence or absence of the object; and when the object detection information indicates that the object is not present, determine whether the estimated posture of subject 11 matches the specific posture.

According to the above features, it is possible to distinguish between a posture that is kept by intervention of an object and a posture that is kept without intervention of an object, to determine whether the estimated posture matches a specific posture. Accordingly, it is possible to estimate the fatigue level of a subject through appropriate computing.

For example, in a blank period which is a period in the predetermined time period and in which the information output device (imaging device 101, for instance) is unable to output the information, estimation device 100 may accumulate a preset supplementary fatigue level in accordance with a length of the blank period.

According to the above feature, even in the case where subject 11 is not included in any of images and fatigue level estimation cannot be carried out, it is possible to perform supplementation using a supplementary fatigue level determined in advance, thereby enabling more accurate estimation of a fatigue level accumulated in a predetermined time period. Accordingly, it is possible to estimate the fatigue level of a subject through appropriate computing.

For example, the specific posture of subject 11 may be defined using a joint position model defined by the relative positions of the joints of subject 11. Estimation device 100 may output the joint position model as a result of the estimation of the posture of subject 11 in the predetermined time period.

According to the above features, it is possible to determine whether an estimated posture matches a specific posture by performing matching between joint position models constructed based on simple information. Accordingly, it is possible to estimate the fatigue level of a subject through appropriate computing.

A fatigue estimation method according to the present embodiment includes: obtaining information regarding the locations of the body parts of subject 11; reading postural fatigue information from a storage device (storage 24), where the postural fatigue information is information in which a specific posture of subject 11 is associated with a unit fatigue level, and the unit fatigue level indicates the level of fatigue accumulated in subject 11 as a result of subject 11 keeping the specific posture for a unit time; estimating the fatigue level of subject 11 in a predetermined time period, where the fatigue level indicates the level of fatigue accumulated in subject 11; estimating the posture of subject 11 in the predetermined time period based on the information output in the predetermined time period; determining whether the estimated posture of subject 11 matches the specific posture; and estimating, as the fatigue level of subject 11 in the predetermined time period, a calculated value obtained by the unit fatigue level being accumulated in accordance with a time period in which the estimated posture of subject 11 is determined to match the specific posture.

With the fatigue estimation method described above, it is possible to obtain the same advantageous effects as those obtained by fatigue estimation system 200 described above.

Moreover, it is possible to implement the present embodiment as a non-transitory computer-readable recording medium having recorded thereon a program for causing a computer to execute the fatigue estimation method described above.

Accordingly, it is possible, with the use of a computer, to obtain the same advantageous effects as those obtained by the fatigue estimation method described above.

### Other Embodiments

Although an embodiment of the present disclosure is described above, the present disclosure is not limited to the embodiment.

For example, in the above embodiment, a process executed by a specific processing unit may be executed by another processing unit. An order of processes may be changed or processes may be executed in parallel.

The fatigue estimation system according to the present disclosure may be implemented by a plurality of devices each having one or more of the components of the fatigue estimation system or by a single device having all of the components. Likewise, the estimation device according to the present disclosure may be implemented by a plurality of devices each having one or more of the components of the estimation device or by a single device having all of the components. One or more of the functions of a component may be implemented as one or more functions of another component, or each of the functions may be distributed to any of components in any way. Any form with a configuration substantially including all of the functions achievable by the fatigue estimation system or the estimation device according to the present disclosure is included in the scope of the present disclosure.

In the above embodiment, the respective components may be implemented by executing software programs suited to the respective components. The respective components may be implemented by a program execution unit such as a CPU or a processor reading and executing a software program recorded on a recording medium such as a hard disk or semiconductor memory.

The respective components may be implemented by hardware. For example, the respective components may be circuits (or integrated circuits). These circuits may compose a single circuit as a whole or may be separate circuits. These circuits may be general-purpose or dedicated circuits.

General or specific aspects of the present disclosure may be implemented using a system, a device, a method, an integrated circuit, a computer program, or a computer-readable recording medium such as a CD-ROM, or any combination of systems, devices, methods, integrated circuits, computer programs, and recording media.

It is also possible to implement, as a method for estimating the posture of a subject, the present disclosure by a configuration that uses a location sensor besides a configuration that uses an imaging device. Specifically, the posture of a subject is estimated using a sensor module including a location sensor and a voltage sensor. Although it is described herein that a plurality of sensor modules are worn by a subject, the number of sensor modules to be worn by a subject is not particularly limited. Only one sensor module may be worn by a subject.

How to wear sensor modules is not particularly limited and any way may be allowed as long as the location of a predetermined body part of a subject can be measured. For example, a plurality of sensor modules are worn by a subject wearing clothing to which the plurality of sensor modules are attached.

A sensor module is a device that is worn by a subject on a predetermined body part and outputs information indicating a detection or measurement result in a manner linked to the predetermined body part. Specifically, the sensor module includes: a location sensor that outputs location information regarding the spatial location of the predetermined body part of the subject; and a voltage sensor that outputs potential information indicating an electric potential at the predetermined body part of the subject. Although a sensor module including both a location sensor and a voltage sensor is exemplified here, a voltage sensor is not essential if a sensor module includes a location sensor. A location sensor in such a sensor module is one example of an information output device that outputs information regarding the locations of the body parts of a subject. Accordingly, the information to be output is location information and includes the relative or absolute location of a predetermined body part of the subject. The information to be output may include, for example, potential information. The potential information is information including the value of an electric potential measured at a predetermined body part of the subject. Hereinafter, the location information and the potential information will be described in detail together with a location sensor and a voltage sensor.

A location sensor is a detector that detects the relative or absolute spatial location of a predetermined body part of a subject on which a sensor module is worn, and outputs information regarding the spatial location of the predetermined body part as the detection result. The information regarding the spatial location includes: information that can identify the location of a body part in a space, as described above; and information that can identify a change in the location of the body part resulting from body movement. Specifically, the information regarding the spatial location includes the locations of joints and bones in a space and information indicating changes in the locations.

A location sensor is composed by combining various sensors such as an acceleration sensor, an angular velocity sensor, a geomagnetic sensor, and a ranging sensor. Since location information output by the location sensor can be approximated to the spatial location of a predetermined body part of a subject, it is possible to estimate the posture of the subject from the spatial location of the predetermined body part.

A voltage sensor is a detector that measures an electric potential at a predetermined body part of a subject on which a sensor module is worn, and that outputs potential information indicating the electric potential at the predetermined body part as the measurement result. The voltage sensor is measuring equipment that includes electrodes and measures a potential generated between the electrodes using an electrometer. The potential information output by the voltage sensor indicates a potential generated at the predetermined body part of the subject. Since the potential corresponds to, for instance, the active potential of muscles in the predetermined body part, it is possible to enhance estimation accuracy in estimating the posture of the subject from, for instance, the active potential of the predetermined body part.

The fatigue estimation system according to one aspect of the present disclosure described herein estimates the fatigue level of a subject using the posture of the subject estimated as described above. Since the processes following the estimation of the posture of the subject are the same as those described in the above embodiment, description thereof is omitted.

The present disclosure may be implemented as a fatigue estimation system or a fatigue estimation method to be executed by an estimation device. The present disclosure may be implemented also as a program for causing a computer to execute such a fatigue estimation method, or as a non-transitory computer-readable recording medium having such a program recorded thereon.

Various modifications to the embodiments which may be conceived by those skilled in the art, as well as embodiments resulting from arbitrary combinations of elements and functions from different embodiments are included within the scope of the present disclosure so long as they do not depart from the essence of the present disclosure.

Reference Signs List

| | |
|---|---|
| 11 | subject |
| 11a, 11c | joint position model |
| 24 | storage (storage device) |
| 100 | estimation device |
| 101 | imaging device (information output device) |
| 200 | fatigue estimation system |

1. A fatigue estimation system comprising:
an information output device that outputs information regarding locations of body parts of a subject;
a storage device which stores postural fatigue information, the postural fatigue information being information in which a specific posture of the subject is associated with a unit fatigue level, the unit fatigue level indicating a level of fatigue accumulated in the subject as a result of the subject keeping the specific posture for a unit time; and
an estimation device that estimates a fatigue level of the subject in a predetermined time period, the fatigue level indicating a level of fatigue accumulated in the subject, wherein
the estimation device:
estimates a posture of the subject in the predetermined time period based on the information output in the predetermined time period;
determines whether the estimated posture of the subject matches the specific posture; and
estimates, as the fatigue level of the subject in the predetermined time period, a calculated value obtained by the unit fatigue level being accumulated in accordance with a time period in which the estimated posture of the subject is determined to match the specific posture.

2. The fatigue estimation system according to claim 1, wherein
in the postural fatigue information:
the specific posture of the subject is associated with a first unit fatigue level that is part of the unit fatigue level, the first unit fatigue level indicating a level of fatigue accumulated in a first part among the body parts of the subject as a result of the subject keeping the specific posture for a unit time; and
the specific posture of the subject is associated with a second unit fatigue level that is part of the unit fatigue level, the second unit fatigue level indicating a level of fatigue accumulated in a second part among the body parts of the subject as a result of the subject keeping the specific posture for a unit time, and
the estimation device:
estimates, as a fatigue level of the first part of the subject in the predetermined time period, a calculated value obtained by the first unit fatigue level being accumulated in accordance with the time period in which the estimated posture of the subject is determined to match the specific posture; and
estimates, as a fatigue level of the second part of the subject in the predetermined time period, a calculated value obtained by the second unit fatigue level being accumulated in accordance with the time period in which the estimated posture of the subject is determined to match the specific posture.

3. The fatigue estimation system according to claim 1, wherein
when determining that the estimated posture of the subject matches the specific posture, the estimation device:
obtains a feature of the estimated posture of the subject;
corrects the unit fatigue level using the feature; and
estimates, as the fatigue level of the subject in the predetermined time period, a calculated value obtained by the corrected unit fatigue level being accumulated in accordance with the time period in which the estimated posture of the subject is determined to match the specific posture.

4. The fatigue estimation system according to claim 2 , wherein
when determining that the estimated posture of the subject matches the specific posture, the estimation device:
obtains a feature of the estimated posture of the subject;
corrects the unit fatigue level using the feature; and
estimates, as the fatigue level of the subject in the predetermined time period, a calculated value obtained by accumulating the corrected unit fatigue level in accordance with the time period in which the estimated posture of the subject is determined to match the specific posture,
the feature is a pressure value obtained from a pressure sensor with which a body part of the subject corresponding to the first part of the subject is in contact, and
the estimation device corrects the first unit fatigue level with a greater amount of correction as the pressure value increases.

5. The fatigue estimation system according to claim 3, wherein
the feature is a difference between the estimated posture of the subject and a reference posture included in a range within which the specific posture falls, and the unit fatigue level is corrected with a greater amount of correction as the difference increases.

6. The fatigue estimation system according to claim 1 , wherein
in the postural fatigue information, the specific posture of the subject is defined as a posture kept by intervention of an object, and
the estimation device:
obtains object detection information indicating presence or absence of the object; and
when the object detection information indicates that the object is present, determines whether the estimated posture of the subject matches the specific posture.

7. The fatigue estimation system according to claim 1 , wherein
in the postural fatigue information, the specific posture of the subject is defined as a posture kept without intervention of an object, and
the estimation device:
obtains object detection information indicating presence or absence of the object; and
when the object detection information indicates that the object is not present, determines whether the estimated posture of the subject matches the specific posture.

8. The fatigue estimation system according to claim 1 , wherein
in a blank period which is a period in the predetermined time period and in which the information output device is unable to output the information, the estimation device accumulates a preset supplementary fatigue level in accordance with a length of the blank period.

9. The fatigue estimation system according to claim 1 , wherein
the specific posture of the subject is defined using a joint position model defined by relative positions of joints of the subject, and
the estimation device outputs the joint position model as a result of the estimation of the posture of the subject in the predetermined time period.

10. A fatigue estimation method comprising:
obtaining information regarding locations of body parts of a subject;
reading postural fatigue information from a storage device, the postural fatigue information being information in which a specific posture of the subject is associated with a unit fatigue level, the unit fatigue level indicating a level of fatigue accumulated in the subject as a result of the subject keeping the specific posture for a unit time;
estimating a fatigue level of the subject in a predetermined time period, the fatigue level indicating a level of fatigue accumulated in the subject;
estimating a posture of the subject in the predetermined time period based on the information output in the predetermined time period;
determining whether the estimated posture of the subject matches the specific posture; and
estimating, as the fatigue level of the subject in the predetermined time period, a calculated value obtained by the unit fatigue level being accumulated in accordance with a time period in which the estimated posture of the subject is determined to match the specific posture.

11. A non-transitory computer-readable recording medium having recorded thereon a program for causing a computer to execute the fatigue estimation method according to claim 10.
